(19) United States
(12) Patent Application Publication
(10) Pub. No.: US 2003/0023232 A1
(43) Pub. Date: Jan. 30, 2003

(54) Determining a target optical zone for ablation based on stiles-crawford effect

(76) Inventor: Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 10/207,116

(22) Filed: Jul. 30, 2002

Related U.S. Application Data
(60) Provisional application No. 60/308,127, filed on Jul. 30, 2001.

(51) Int. Cl. A61B 18/18; A61B 19/00

(52) U.S. Cl. 606/5; 128/898; 606/10

(57) ABSTRACT

The optimal optical zone for a patient is determined based upon the Stiles-Crawford effect and patient-specific scotopic pupil parameters. This invention is especially useful in refractive surgery where the chosen optical zone is crucial and affects both the depth and volume of the ablation. This optimal optical zone may be used in the creation of corrective lenses and other ocular surgery in addition to refractive surgery. Provided with the optimal optical zone, a refractive surgeon may better choose the surgical parameters for the treatment. Current methods consist of choosing a default value for all patients or only choosing the scotopic pupil diameter or contour, either of which may not be optimal and may lead to a deeper or bigger treatment.

This application is based on and claims priority from U.S. Provisional Application No. 60/308,127 filed on Jul. 30, 2001, the entirety of which is expressly incorporated herein by reference.

### BACKGROUND OF THE INVENTION

1. Field of the Invention

The invention relates to corneal refractive surgery and, more particularly, to a method of determining the patient-specific optimal optical zone based on the Stiles-Crawford effect and the patient scotopic pupil parameters (such as diameter).

2. Background

For corneal refractive surgery, an important feature is the optical zone of the treatment. The optical zone is the annular area of the ablated surface (although not necessarily circular, it may be elliptical or have some other shape corresponding to the pupil shape) that ideally follows the prescription. The treatment zone (also called the blending or transition zone because it should provide a smooth transition to the eye surface) encompasses the optical zone and is generally a function (usually a constant offset or a curvature gradient) of the optical zone.

The Stiles-Crawford effect is an optical phenomenon of the eye where rays that enter the pupil near the center are more effective (appear brighter) than oblique rays (near the periphery of the pupil). This means that not all rays that enter the pupil are equally effective, and it leads us to conclude that not all the rays entering the pupil are useful.

Current methods for choosing the optical zone are based upon the scotopic pupil diameter, a constant value (such as 6.0 mm diameter), ablation depth, ablation volume, or similar parameters. These are all reasonable choices, but may not provide the most optimal solution for the patient. A choice based upon the individual characteristics of the patient's eye may provide the best result.

### SUMMARY OF THE INVENTION

An object of the invention is to determine the optimal optical zone for the patient based upon the scotopic pupil parameters of the patient and the Stiles-Crawford effect. The optimal optical zone is derived from a function expressed in terms of the Stiles-Crawford coefficient β and the scotopic pupil parameters (such as diameter).

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 is a plan view showing an exemplary scotopic pupil contour 10. It also shows an exemplary scotopic pupil diameter 15 and exemplary optimal optical zone 20 determined in accordance with this invention.

FIG. 2 is a graph illustrating some exemplary values of the Stiles-Crawford coefficient, as used in accordance with this invention.

FIG. 3 is a flowchart of a method of the present invention determining the optimal optical zone using the Stile-Crawford effect and the scotopic pupil parameter.

### DETAILED DESCRIPTION OF ILLUSTRATIVE EMBODIMENTS

Each human cornea has a window through which the person sees. This aperture is called the pupil. If a person has poor vision that can be corrected by refractive surgery, then the size of the ablated area that covers the pupil is an important surgical parameter. This invention relates to refractive surgery, which works by removing material from the cornea to shape it into a surface that corrects for this refractive focal distance error.

An important consideration in this surgery is determining how large an area the ablation should affect. The ablated area that contains the unaltered prescription for the patient is called the optical zone. A further transition zone extends beyond the optical zone and is primarily used to provide a smooth blend to the cornea. It is not expected that the patient is able to see well through the transition zone, in the event that the transition zone covers part of the pupil. Thus, the size and shape of the optical zone becomes a crucial parameter to the surgical plan.

The pupil is not necessarily circular. In general, it can be elliptical or hold some other freeform shape (likely expressed as a collection of data points). Thus, the scotopic pupil parameter or parameters could be anything that describes the shape of the pupil. The primary choice, however, will be the scotopic pupil diameter because it is easily used (a single value) and will probably be the maximum value encompassing the entire pupil. Unless the pupil is very oddly shaped, using the scotopic pupil diameter will waste little extra tissue. Thus, the examples and further detailed description below utilize only the scotopic pupil diameter.

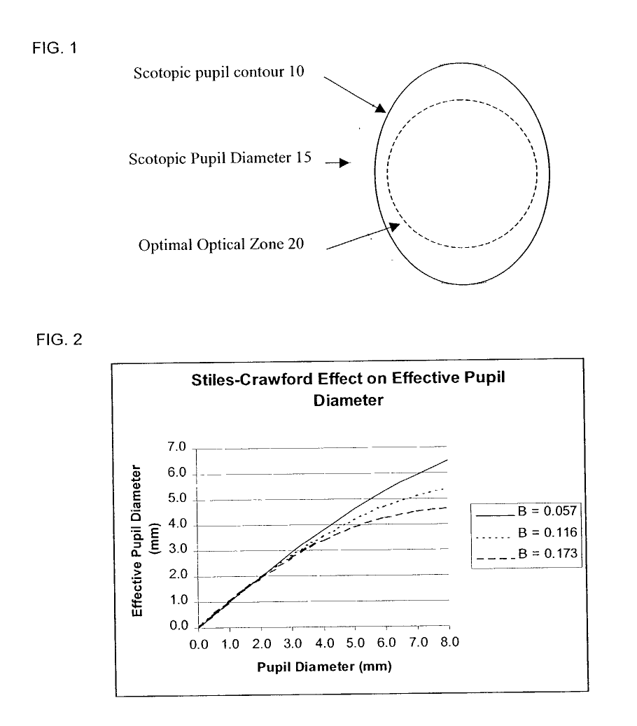

FIG. 1 is an example representation of the method of this invention. The scotopic pupil contour 10 may be circular, elliptical, or some other freeform shape. Generally, the method of acquiring the scotopic pupil contour determines its shape and the parameters describing its shape. Some devices used in acquiring the scotopic pupil contour are a corneal topographer, pupillometer, and wavefront analyzer. The parameter for the scotopic pupil chosen for this example is the maximum scotopic pupil diameter and a circle representing that value is given in 15. This value could have been the actual contour (likely represented as a set of points, or optionally as a polynomial or parametric curve), or an ellipse. However, both of these alternative representations would create a far more complex function in determining the optimal optical zone and would not enhance the example. The optimal optical zone based upon that parameter and the method described herein is shown as 20, and, in this example case, is also circular. In this example, the optimal optical zone is smaller than the scotopic pupil diameter, representing the likelihood that such will usually be the case. The optimal optical zone will rarely be larger than the scotopic pupil because ablation outside the aperture of the eye provides no additional refractive power. The optimal optical zone is frequently the same size as the scotopic pupil.

An example equation for use with this method, given the scotopic pupil diameter follows:

$${D}_{O Z} = \frac{4 [ 1 - exp ( - β {D}_{S} / 4 ) ]}{β {D}_{S}^{2}}$$

In the above equation, D_{S} is the scotopic pupil diameter, β is the Stiles-Crawford coefficient, and D_{OZ} is the optimal optical zone. Some examplary values for the Stiles-Crawford coefficient are published in, e.g., Atchison, David A. and George Smith, “Optics of the Human Eye”, Butterworth-Heinemann, Oxford (2000), and are reproduced in part for convenience in FIG. 2. The practitioners of this invention may substitute additional work and thus their own value, or values, for the coefficient. Note that the equation could be more complex and thus require additional coefficients, depending on the scotopic pupil parameters chosen. Although the clinician may determine his or her own coefficient based upon some population, it is not recommended that the determination of the coefficient be done with a population of patients containing any retinal pathology.

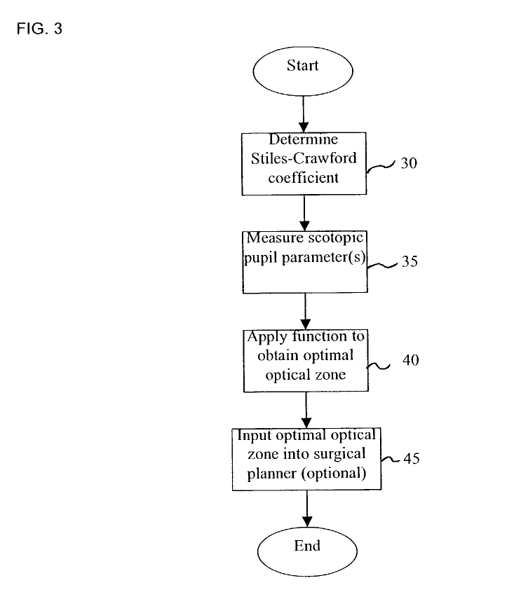

FIG. 3 is an exemplary flowchart of a method of the present invention. The method steps of FIG. 3 are preferably implemented in appropriate software, e.g., in the surgical planner of the laser system or secondary computer. In particular, with reference to step 30 of FIG. 3, the Stiles-Crawford coefficient(s) is determined. As noted above, this can be from existing data or from newly derived data.

In step 35, the patient's scotopic pupil is measured and the appropriate parameter(s), e.g. maximum scotopic pupil diameter in reference to FIG. 1, are obtained.

In step 40, the parameter(s) are input into the function with the Stiles-Crawford coefficient(s) to determine the optimal optical zone. The output of this function can be as simple as the circular optical zone diameter or ellipse parameters (major axis and length, minor axis and length), or as complicated as a set of optical zone data points expressing a freeform curve or even a polynomial or parametric curve equation.

In step 45, the optimal optical zone is input into a surgical planner, which typically resides on a computer as a software program. This computer can be located in the laser system, in the measurement device (e.g. topographer), or as a stand-alone computer system. This step is optional and might not be used if the purpose of the apparatus built according to the present invention is purely diagnostic.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of determining an optimal optical zone for ablation, said method comprising:
determining at least one Stiles-Crawford coefficient;
measuring at least one parameter of a scotopic pupil; and
deriving an optimal optical zone for ablation based on a function including both said Stiles-Crawford coefficient and said parameter of a scotopic pupil.

2. The method of determining an optimal optical zone for ablation according to claim 1, further comprising:
inputting said optimal optical zone into a surgical planner.

3. The method of determining an optimal optical zone for ablation according to claim 1, wherein:
said parameter of said scotopic pupil relates to a diameter of said scotopic pupil.

4. The method of determining an optimal optical zone for ablation according to claim 3, wherein:
said parameter of said scotopic pupil is a diameter of said scotopic pupil.

5. The method of determining an optimal optical zone for ablation according to claim 3, wherein:
said parameter of said scotopic pupil is a radius of said scotopic pupil.

6. The method of determining an optimal optical zone for ablation according to claim 1, wherein said parameter of said scotopic pupil comprises:
an elliptical contour of said scotopic pupil, said elliptical contour comprising:
a major axis and length, and
a minor axis and length.

7. The method of determining an optimal optical zone for ablation according to claim 1, wherein said parameter of said scotopic pupil comprises:
a contour of said scotopic pupil, said contour comprising:
a freeform surface represented by a set of points.

8. The method of determining an optimal optical zone for ablation according to claim 1, wherein said parameter of said scotopic pupil comprises:
a contour of said scotopic pupil, said contour comprising a curve represented by at least one of:
a polynomial equation; and
at least one parametric equation.

9. Apparatus for determining an optimal optical zone for ablation, comprising:
means for determining at least one Stiles-Crawford coefficient;
means for measuring at least one parameter of a scotopic pupil; and
means for deriving an optimal optical zone for ablation based on a function including both said Stiles-Crawford coefficient and said parameter of a scotopic pupil.

10. The apparatus for determining an optimal optical zone for ablation according to claim 9, further comprising:
means for inputting said optimal optical zone into a surgical planner.

11. The apparatus for determining an optimal optical zone for ablation according to claim 9, wherein:
said parameter of said scotopic pupil relates to a diameter of said scotopic pupil.

12. The apparatus for determining an optimal optical zone for ablation according to claim 11, wherein:
said parameter of said scotopic pupil is a diameter of said scotopic pupil.

13. The apparatus for determining an optimal optical zone for ablation according to claim 11, wherein:
said parameter of said scotopic pupil is a radius of said scotopic pupil.

14. The apparatus for determining an optimal optical zone for ablation according to claim 9, wherein said parameter of said scotopic pupil comprises:
an elliptical contour of said scotopic pupil, said elliptical contour comprising:
a major axis and length, and
a minor axis and length.

15. The apparatus for determining an optimal optical zone for ablation according to claim 9, wherein said parameter of said scotopic pupil comprises:
a contour of said scotopic pupil, said contour comprising:
a freeform surface represented by a set of points.

16. The apparatus for determining an optimal optical zone for ablation according to claim 9, wherein said parameter of said scotopic pupil comprises:
a contour of said scotopic pupil, said contour comprising a curve represented by at least one of:
a polynomial equation; and
at least one parametric equation.
